(19) United States
(12) Patent Application Publication
(10) Pub. No.: US 2022/0306505 A1
(43) Pub. Date: Sep. 29, 2022

(54) A SIMPLE METHOD FOR DESICCATION AND REACTIVATION OF AEROBIC GRANULES

(71) Applicant: NUGRAN BIOTECH PTE. LTD., Tanjong Pagar (SG)

(72) Inventors: Joo Hwa Tay, Calgary (CA); Shun Ogura, Niigata (JP); Rania Ahmed Hamza Sayed Eid, Calgary (CA); Zhiya Sheng, Calgary (CA)

(21) Appl. No.: 17/615,521

(22) PCT Filed: May 29, 2020
(86) PCT No.: PCT/CA2020/050736

§ 371 Date: Nov. 30, 2021

Related U.S. Application Data
(60) Provisional application No. 62/855,312, filed on May 31, 2019.

(51) Int. Cl. C02F 3/34; C02F 3/12; C12N 1/20; C12N 1/04; F26B 5/00

(52) U.S. Cl. CPC C02F 3/348; C02F 3/12; C12N 1/20; C12N 1/04; F26B 5/005; C02F 2003/001

(57) ABSTRACT

There is described here a method of desiccation of aerobic granules.

### CROSS REFERENCE TO RELATED APPLICATION

This application claims priority to U.S. Provisional patent application US 62/855,312, filed May 31, 2019; the entire contents of which is hereby incorporated by reference.

### FIELD

The present disclosure relates generally to a method of desiccation of aerobic granules.

### BACKGROUND

Aerobic granules may be used in the treatment of wastewaters. Generally, aerobic granules develop from flocculent sludge to compact aggregates, granular sludge, and finally mature aerobic granules. Transportation of the seed granules is costly and time-consuming, especially when the seed granules are to be transported to another country.

There remains a need to produce desiccated aerobic granules.

### SUMMARY

In one aspect there is provided a method of producing desiccated aerobic granules, comprising:

(a) subjecting aerobic granules to a size selecting step;

(b) subjecting the size selected aerobic granules from (a) to a dehydration step;

(c) subjecting the dehydrated size selected aerobic granules from (b) to a drying step.

In one example,

In one example, said size selection comprises passing said aerobic granules through a filter.

In one example, said filter is a sieve.

In one example, said filer comprises a plurality of prose with a diameter of about 1 mm.

In one example, said dehydration step comprises contacting said size selected aerobic granules with an organic dehydration solution.

In one example, said dehydration solution comprises or consists of acetone:isopropanol (95%:5% v/v).

In one example, drying step comprises spin drying the dehydrated size selected aerobic granules.

In one example, said drying step is at room temperature.

In one example, said aerobic granule is a mature aerobic granule.

In one aspect there is provided desiccated aerobic granules produced according to the method of any one of claims 1 to 9.

In one aspect there is provided a method of wastewater treatment, comprising: adding desiccated aerobic granules of claim 10, to a wastewater treatment plant.

In one aspect there is provided a kit comprising: the desiccated aerobic granules of claim 10, and a container, and optionally instructions for the use thereof.

### BRIEF DESCRIPTION OF THE FIGURES

Embodiments of the present disclosure will now e described, by way of example only, with reference to the attached Figures.

FIG. 1 is a flow chart of a method of the present application.

FIG. 2 is the schematic diagrams of the drying machines: (a) air-drying; (b) oven-drying; (c) sun-drying

FIG. 3 shows drying process granules: (a) sieving; (b) dehydrated granules; and (c) granules after drying.

FIG. 4 shows reactivated granules after two hours.

FIG. 5 depicts recovery of granule activity: (a) COD (b) Ammonia nitrogen; and (c) phosphorus.

FIG. 6 depicts unsuccessful products: (a) granules dried without the drying device; and (b) air dried granules with no treatment.

### DETAILED DESCRIPTION

Aerobic granules are compact aggregates of microorganisms that are self-immobilized without the need for carrier media. Aerobic granular sludge process is more and more recognized as a promising wastewater treatment technology which has shown its advantages in high treatment capacity with significantly reduced plant footprint and operational costs. However, the relatively long start-up phase remains one of the major challenges for this novel technology. Transportation of the seed granules is costly and time-consuming, especially when the seed granules are to be transported to another country.

There is described herein a method to dry aerobic granules. In some examples, the desiccation process only takes a few hours. The dried granules are easier to transport and can be stably stored for at least five months. The desiccation process was designed to be scalable and to maximally maintain the microbial community in aerobic granules, especially the major functional groups, so that both COD and nutrient removal abilities can be preserved. It has been verified that structural stability and biological activity can be recovered within 24 hours after the desiccation and storage process.

Aerobic granular sludge technology has been paid attention to as a promising wastewater treatment system. Compared to the conventional system, the methods described herein exhibits outstanding settling, simultaneous removal of organic substrates and nutrients in the same tank, which leads to a smaller footprint, lower capital and operational cost and higher removal efficiency. One of the challenges impeding the full-scale application of the technology in real wastewater plants is a long time required to cultivate the granules. As a solution, it is suggested that granules be cultivated and stored outside the reactor and used to inoculate the reactor again as the need arises.

The key point of storing granule is prolonging the granule stability by preventing the activity of proteolytic bacteria not to cause intragranular protein hydrolysis. Most microbial activities in dried granules cease so prolonged storage is possible. Additionally, dried granules should be recoverable in a short time and with minimal loss of structural integrity. The drying process makes it easy to store and transport granules since fresh sludge contains large amount of water (>99%), and dehydrating leads to volume and weight reduction. In one example, described herein is a new air-drying method for drying of aerobic granules.

### Drying Process

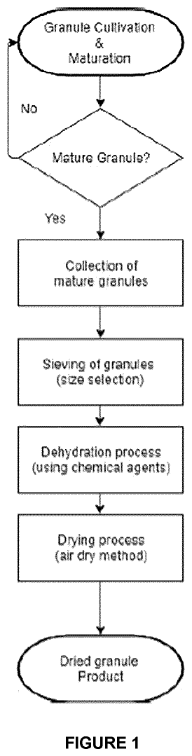

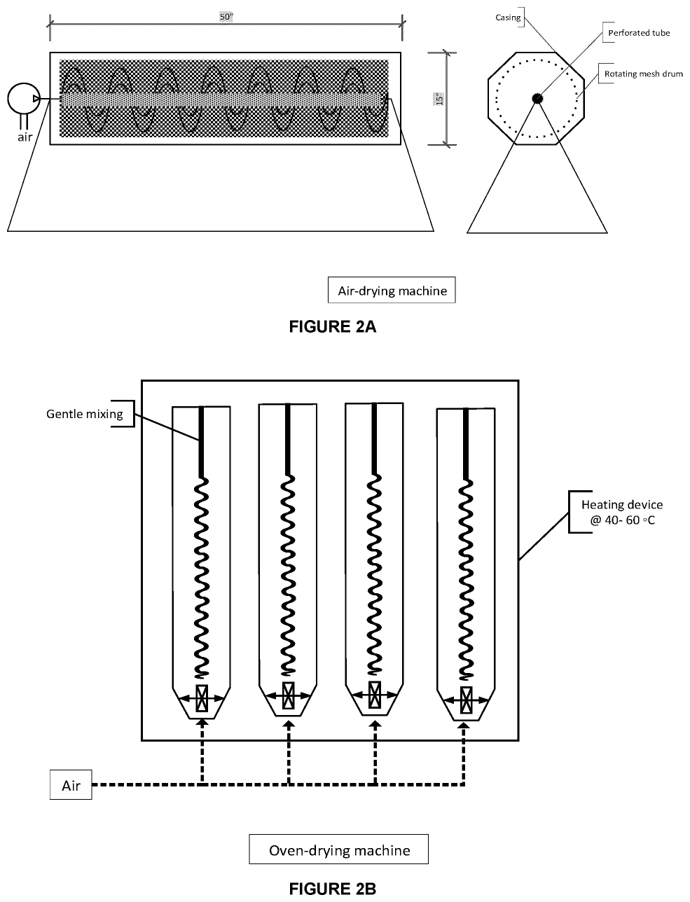

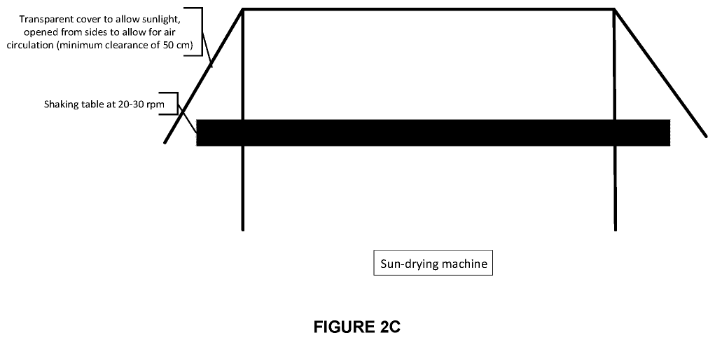

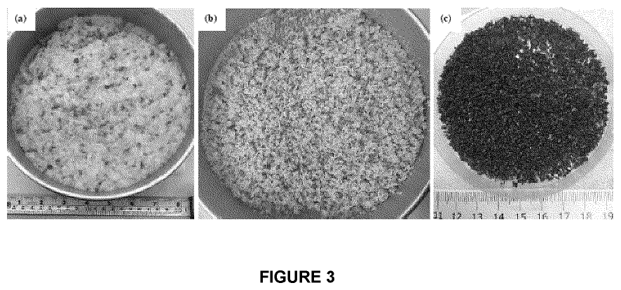

A cylindrical acrylic reactor of an effective volume of 20 L and internal diameter of 15 cm was used to cultivate the aerobic granules. Mature granules were sieved at 1 mm sieves and soaked in the dehydration solution (acetone:isopropanol=95:5 v/v) to dehydrate the surface of granules for 2 hours. Afterwards, the granules are dried in the drying device under room temperature. Process flowchart is shown in FIG. 1. Average dehydration rate of dried granules was ˜98% (i.e. weight reduction of 98%). A schematic diagram of the air-drying machine is shown in FIG. 2A. The granules throughout the drying process is shown in FIG. 3. The drying machine incorporates a rotating drum which facilitates drying without clumping, with a mesh size of 0.2 mm.

### Dried Granules Reactivation

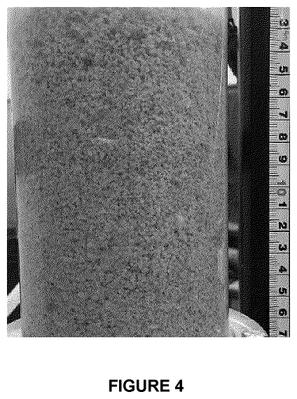

The dried granules were reinoculated, after over five months of storage, to test the activation of the biomass and the treatment performance in terms of organics and nutrients removal. After 18 hours of inoculation, the dried granules were proven to resume their metabolism after going through the dehydration and air-dried process, as shown in FIG. 4.

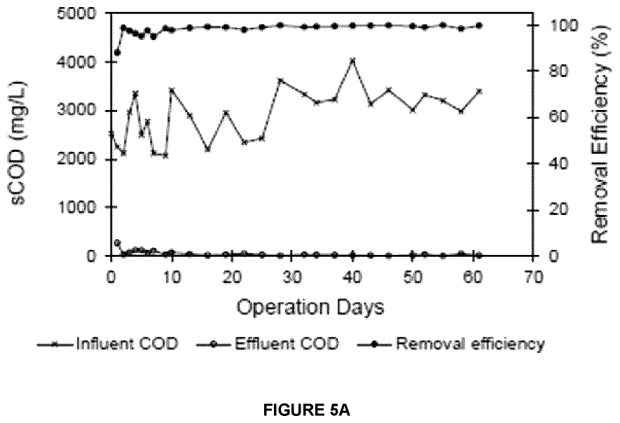

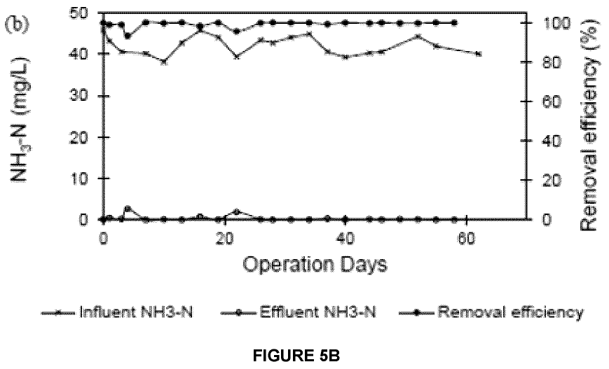

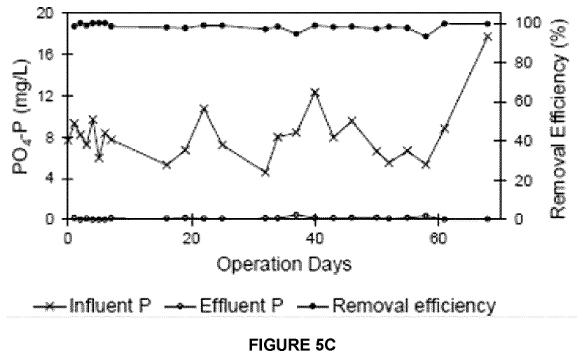

The profiles of COD, ammonia and phosphorus removals are shown in FIG. 5. After one day of inoculation, COD removal achieved over 90% and removal remained stable and satisfactory for the entire operation period of two months, even when an influent COD concentration over 3000 mg/L was tested. Ammonia nitrogen removal rate was over 90% from the very beginning and was over 99% most of the time during the two-month test. Good phosphorus removal was achieved throughout the test period as well.

In one example there is described a method of producing desiccated aerobic granules, comprising:

(a) subjecting aerobic granules to a size selecting step;

(b) subjecting the size selected aerobic granules from (a) to a dehydration step;

(c) subjecting the dehydrated size selected aerobic granules from (b) to a drying step.

In one example, said size selection comprises passing said aerobic granules through a filter.

In one example, said filter is a sieve. It will be appreciated that a variety of types of filters may be used, as long as the propose size may be selected. In a specific example, the pore size of the filter is about 0.2 mm.

In one example, said filer comprises a plurality of pores with a diameter of about 1 mm.

In one example, said dehydration step comprises contacting said size selected aerobic granules with an organic dehydration solution.

In one example, said dehydration solution comprises or consists of acetone:isopropanol (95%:5% v/v).

In one example, drying step comprises spin drying the dehydrated size selected aerobic granules. In some examples, the drying step may be carried out with a loading of about 20 L and a speed of about 5-20 rpm.

In one example, said drying step is at room temperature. In other examples, said drying step is between about 15° C. to about 60° C.

In some examples, drying may be sun-drying, air-drying or oven-drying.

In one example air-drying is performed using the designed apparatus shown in FIG. 2A. The drying machine incorporates a rotating drum which facilitates drying without clumping, with a mesh size of 0.2 mm.

Oven-dry may be carried out under temperatures up to 60° C. A columnar reactor module(s) is designed where gentle mixing using a propeller at 20-30 rpm is used in addition to upflow air blowing as shown in FIG. 2B. The column module is a versatile design that can offer flexibility in the volume of granules to be dried. To promote faster drying a column of diameter 8-15 cm can be used with height to diameter ratio of 2-5.

In one example, to dry with sunlight, a flat surface with a transparent cover at minimum clearance of 50 cm is required. Size screening is an important step before drying. Screened granules are laid flat on a shaking table at 20-30 rpm to avoid granules clumping (a schematic of the drying table is shown in FIG. 2C.)

In one example, said aerobic granule is a mature aerobic granule.

In one aspect there is desiccated aerobic granule produced according to the method of any one of claims 1 to 9.

In one aspect, there is described a method of wastewater treatment, comprising: adding a desiccated aerobic granule of claim 10, to a wastewater sample comprising a biomass.

The desiccated granules can be used to start up a new aerobic granular reactor for wastewater treatment or added to existing wastewater treatment plants as augmenting reagent. No special rehydrating process is required for the application of the desiccated granules. Compared to existing inoculation method with fresh granules, the storage, transportation and addition process become much easier with the desiccated granules and much shorter time will be required to reactivate the granules.

In one aspect there is described a kit comprising: the desiccated aerobic granule of claim 10, and a container, and optionally instructions for the use thereof.

Comparison with Previous or Unsuccessful Drying Methods

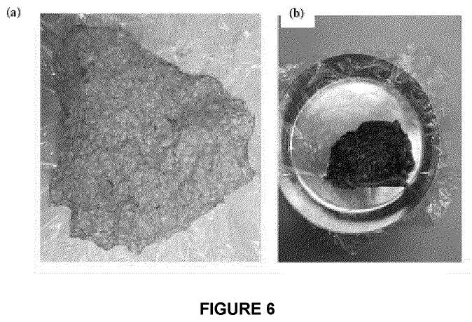

The inventors have published a few methods for drying granules in small quantities. Compared to previously published methods, advantages of this invention include: 1) Removal of manual surface drying process. Most of the published methods include a step of initial removal of surface moisture by adsorbent papers. However, this step can easily deteriorate granule integrity and it does not work for large-quantity practice in the industry. This step was removed from the current procedure. 2) Simplification of dehydration process. The chemical methods published so far usually involves the use of a series of dehydration solutions with gradually increased organic portion, which is time and manpower consuming if applied in the industry. Efforts were put into the invention to simplify the method and to make it feasible for large-quantity application. Various dehydration solutions were tested and the best one for the simplified process was selected, which is also the recipe with the lowest number of ingredients among solutions with similar performance. 3) Ideal weight and volume reduction. Previously published methods achieved only partial drying and no further drying steps were taken after the chemical dehydration. A complete desiccation can be achieved in the current invention and that is to say more weight and volume reduction can be reached which leads to more savings on transportation cost. 4) No clump formation after complete drying. The combination of the cultivation method and sieving process enables efficient granule selection. Proper selection plus the rotating mesh drum in the drying device enables gentle drying that minimizes physical damage while keeping granules from forming clumps. A comparison was shown in FIG. 6. Without the designed drying device, the granules stuck together and form large clumps. If the granules are air-dried directly without any selection, chemical treatment or the drying device, even more compact clumps were formed with significant shrinking and the granular structure was completely lost. 5) Both COD and nutrient removal capacities can be maintained and quickly recovered. The recovery of nutrient removal capacity of aerobic granules has never been addressed in any previous publications. The combination of all the improvement enables better maintenance of the functional microorganisms in aerobic granules and the diversity of microbial community. Therefore, both COD and nutrient removal abilities can be maintained and quickly recovered. Good nitrogen and phosphorus removal can be recovered within 24 hours, which has never been reported before.

### Examples of Usage of Desiccated Granules

There are at least two types of applications of the desiccated aerobic granules: 1) to augment existing wastewater treatment plants that use the conventional activated sludge technology; 2) to inoculate new wastewater treatment plants.

If used as augmentation, there is no specific additional requirement on the activated sludge bioreactor. The desiccated aerobic granules can be directly added into the existing activated sludge bioreactor as needed.

If used to inoculate new aerobic granular sludge plants, the plant bioreactor has to be either an existing bioreactor using the aerobic granular sludge technology such as a Nereda® reactor or constructed and operated as sequencing batch reactor with a minimum height/diameter ratio of 3, a maximum settling time of 15-20 minutes and fine-bubble air supply from the bottom, Preferred inoculation concentration will be about 5 g desiccated granules per liter of wastewater, but it can be as low as 2-3 g/L depending on the wastewater quality. The reactor size can vary from laboratory scale (as small as 1-2 L) to full-scale plant reactors that can provide treatment capacity over 400,000 m3/day. No specific reactivation step will be required for desiccated aerobic granules. The new reactor can be operated at full-capacity right after inoculation and pollutants removal performance can recover within 24 hours, if the desiccated aerobic granules are cultured in a mother reactor fed with the same wastewater. The desiccated aerobic granules can potentially be used to seed conventional activated sludge plants as well. In that case, ideal inoculation concentration can be 2-3 g desiccated granules per liter of wastewater and there is no special requirements on bioreactor design or operation. Preliminary tests have been done in China before and more tests can be carried out in Canada if needed.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A method of producing desiccated aerobic granules, comprising:
(a) subjecting aerobic granules to a size selecting step;
(b) subjecting the size selected aerobic granules from (a) to a dehydration step;
(C) subjecting the dehydrated size selected aerobic granules from (b) to a drying step.

2. The method of claim 1, wherein said size selection comprises passing said aerobic granules through a filter.

3. The method of claim 2, wherein said filter is a sieve.

4. The method of claim 2 or 3, wherein said filer comprises a plurality of prose with a diameter of about 1 mm.

5. The method of any one of claims 1 to 4, wherein said dehydration step comprises contacting said size selected aerobic granules with an organic dehydration solution.

6. The method of claim 5, wherein said dehydration solution comprises or consists of acetone:isopropanol (95%:5% v/v).

7. The method of any one of claims 1 to 6, wherein drying step comprises spin drying the dehydrated size selected aerobic granules.

8. The method of claim 7, wherein said drying step is at room temperature.

9. The method of any one of claims 1 to 8, wherein said aerobic granule is a mature aerobic granule.

10. Desiccated aerobic granules produced according to the method of any one of claims 1 to 9.

11. A method of wastewater treatment, comprising: adding desiccated aerobic granules of claim 10, to a wastewater treatment plant.

12. A kit comprising: the desiccated aerobic granules of claim 10, and a container, and optionally instructions for the use thereof.
